(19) United States
(12) Patent Application Publication
(10) Pub. No.: US 2015/0073278 A1
(43) Pub. Date: Mar. 12, 2015

(54) OBJECT INFORMATION ACQUIRING APPARATUS AND CONTROL METHOD THEREOF

(71) Applicant: CANON KABUSHIKI KAISHA, Tokyo (JP)

(72) Inventor: Kenji Oyama, Tokyo (JP)

(21) Appl. No.: 14/475,681

(22) Filed: Sep. 3, 2014

(30) Foreign Application Priority Data
Sep. 11, 2013 (JP) 2013-188242

(51) Int. Cl. A61B 5/00; A61B 8/08; A61B 5/107

(52) U.S. Cl. CPC A61B 5/0095; A61B 5/1079; A61B 8/0858; A61B 5/0035; A61B 5/708; A61B 5/742; A61B 8/0825; A61B 5/4312; A61B 5/441
USPC 600/449; 600/407

(57) ABSTRACT

There is used an object information acquiring apparatus including an irradiating unit that irradiates an object with light, an irradiation position controlling unit that controls an irradiation position for irradiating the object with the light, a probe that receives an acoustic wave generated when the object is irradiated with the light from the irradiating unit, at a position opposing the irradiating unit across the object, and outputs an acoustic wave signal, a probe controlling unit that controls the probe, a control processor that controls at least one of the irradiation position controlling unit and the probe controlling unit such that the light does not enter the probe directly without going through the object, and a constructing unit that constructs characteristic information.

### BACKGROUND OF THE INVENTION

1. Field of the Invention

The present invention relates to an object information acquiring apparatus and a control method thereof.

2. Description of the Related Art

There is proposed a technology called photoacoustic tomography (PAT) that acquires functional information on a living body by using a photoacoustic effect. The PAT is proved to be useful especially in the diagnosis of skin cancer and breast cancer, and there are growing expectations for the PAT as medical equipment that replaces an ultrasonic imaging apparatus, an X-ray apparatus, or an MRI apparatus that has been used in the diagnosis thereof.

The photoacoustic effect is a phenomenon in which, when an object such as a body tissue or the like is irradiated with pulsed light such as visible light or near infrared light, a light absorbing material (hemoglobin in blood or the like) in the object absorbs energy of the pulsed light, expands instantaneously, and generates a photoacoustic wave (typically ultrasonic wave). In the PAT, characteristic information on the body tissue (object information) is visualized by measuring the photoacoustic wave.

An example of the object information includes a light energy absorption density distribution indicative of the density distribution of the light absorbing material in the living body that has served as the generation source of the photoacoustic wave. By visualizing the light energy absorption density distribution, it is possible to image active vascularization by a cancer tissue. In addition, by utilizing light wavelength dependence of the generated photoacoustic wave, functional information such as the oxygen saturation of blood or the like is obtained. In addition, it is also possible to acquire information on glucose and cholesterol.

Further, the PAT uses light and the ultrasonic wave for imaging biological information, and hence it is possible to perform image diagnosis in a non-radiation-exposed and noninvasive state, and has a significant advantage in terms of a patient burden. Consequently, instead of the X-ray apparatus having difficulty in repetitive diagnosis, the PAT is expected to be actively involved in screening and early diagnosis of breast cancer.

An initial sound pressure Po of the photoacoustic wave generated as the result of absorption of light by the light absorbing material is calculated by the following Expression:

Po=Γ·μₐ·Φ (1)

wherein Γ represents a Gruneisen coefficient that is obtained by dividing the product of an expansion coefficient β and the square of a sound velocity c by a specific heat at constant pressure Cₚ. It is known that Γ has a substantially constant value depending on the object. μₐ represents a light absorption coefficient of the light absorbing material. Φ represents a light amount in the object, i.e., an amount of light that has actually reached the light absorbing material (light fluence).

By dividing an initial sound pressure distribution Pₒ by the Gruneisen coefficient Γ, it is possible to calculate the distribution of the product of μₐ and Φ, i.e., the light energy absorption density distribution. The initial sound pressure distribution Pₒ is obtained by measuring the change with time of a sound pressure P of the photoacoustic wave that propagates in the object and reaches a probe.

Further, by calculating the distribution of the light amount Φ in the object, it is possible to calculate the distribution of the light absorption coefficient μₐ in the object as the measurement target. Note that light reaches the deep part of the object while being significantly diffused and decayed in the object, and hence the light amount Φ of light that has actually reached the optical absorbing material is calculated from a light decay amount and a reached depth in the object.

According to Expression (1), the initial sound pressure Po depends on the product of the light absorption coefficient μₑ and the light amount Φ. Accordingly, even when the light absorption coefficient has a small value, in the case where the light amount is large, the generated photoacoustic wave is large. In addition, even when the light amount is small, in the case where the light absorption coefficient has a large value, the photoacoustic wave is also large.

Japanese Patent No. 4448189 describes a photoacoustic tomography apparatus having an opposing configuration. The opposing configuration denotes a configuration in which an irradiation opening of pulsed light formed by an irradiation optical system and a probe for detecting the photoacoustic wave oppose each other across the object. The apparatus having the opposing configuration of Japanese Patent No. 4448189 obtains the biological information in an object area positioned at the front of the probe by synchronizing the irradiation of the pulsed light and a reception operation of the photoacoustic wave.

According to the technology of Japanese Patent No. 4448189, even during continuous movement of the probe, it is possible to acquire object information having high reliability. In addition, by successively performing measurement while performing two-dimensional scanning using an acquisition position of the object information on the object, it becomes possible to measure a wide object area even with a small probe.

Note that, in the opposing configuration disclosed in Japanese Patent No. 4448189, when the measurement of the photoacoustic wave is performed at a position where the object is not present, the pulsed light reaches the surface of the probe while maintaining high energy without entering the object. According to Expression (1), even when the light absorption rate of a member constituting the surface of the probe is small, in the case where the reaching light maintains high energy, it follows that the member of the surface of the probe generates a strong photoacoustic wave.

Generally speaking, the intensity of the acoustic wave from the surface of the probe is high as compared with the intensity of the photoacoustic wave from the light absorbing material in the object. Accordingly, there is a possibility that the photoacoustic wave having effective object information is not found.

In addition, the acoustic wave from the surface of the probe is received as a large signal immediately after light irradiation, and hence the decay thereof takes time. Further, the position of generation of the acoustic wave from the surface of the probe is close to the surface of the object, and hence the photoacoustic wave from the object reaches the probe before the acoustic wave from the surface of the probe is decayed, and signals are mixed up. Because of these reasons, it is difficult to temporally separate the acoustic wave from the surface of the probe from the acoustic wave from the inside of the object.

U.S. Patent Application Publication No. 2010/0053618 discloses a technology for preventing the generation of the photoacoustic wave by the surface of the probe by reducing light absorption on the surface of the probe by disposing a reflective member on the surface of the probe.

In the opposing configuration of the apparatus, light emitted to the object reaches the deep part of the object while being significantly diffused and decayed in the object, and a part of the light passes through the object and reaches the surface of the probe. According to the configuration of U.S. Patent Application Publication No. 2010/0053618, the light having reached the surface of the probe while being significantly decayed in the object is further reflected by the reflective member, and hence an effect of suppressing the photoacoustic wave generated on the surface of the probe is obtained.
Patent Literature 1: Japanese Patent No. 4448189
Patent Literature 2: U.S. Patent Application Publication No. 2010/0053618

### SUMMARY OF THE INVENTION

However, in the case where the pulsed light having high energy has reached the surface of the probe without being decayed, the reflectance of the reflective member is about 98% at most, light absorption of about a few percent occurs. As a result, a problem arises in that the reflective member also generates a strong photoacoustic wave.

Further, in the configuration of the apparatus that acquires the object information in a wide region by repeating the measurement while performing two-dimensional scanning using the irradiation opening of the pulsed light and the probe, another problem arises. That is, depending on the scanning position on the object, there are cases where a part of the pulsed light reaches the surface of the probe while maintaining high energy without being decayed. For example, in breast cancer diagnosis in a breast oncology department, it is necessary to measure not only the central part of a breast as an object but also the peripheral edge part thereof. Accordingly, a part of the pulsed light directly goes toward the probe without going through the object, and hence the reflective member disposed on the surface of the probe generates a strong photoacoustic wave.

As described above, when the strong photoacoustic wave is generated from the surface of the probe or the reflective member, the photoacoustic wave becomes a noise when the inside of the object is reconstructed as an image, and the image may become inappropriate for image diagnosis.

The present invention has been achieved in view of the above problems, and an object thereof is to prevent the generation of the photoacoustic wave caused by direct irradiation of light to the probe in the photoacoustic tomography.

The present invention provides an object information acquiring apparatus comprising:

an irradiating unit configured to irradiate an object with light;

an irradiation position controlling unit configured to control an irradiation position for irradiating the object with the light;

a probe configured to receive an acoustic wave generated when the object is irradiated with the light from the irradiating unit, at a position substantially opposing the irradiating unit across the object, and output an acoustic wave signal;

a probe controlling unit configured to control reception of the probe;

a control processor configured to control at least one of the irradiation position controlling unit and the probe controlling unit such that the light does not enter the probe directly without going through the object; and

a constructing unit configured to construct characteristic information on an inside of the object from the acoustic wave signal.

The present invention also provides a object information acquiring apparatus comprising:

an irradiating unit configured to irradiate an object with light;

an irradiation position controlling unit configured to control an irradiation position for irradiating the object with the light;

a probe configured to receive an acoustic wave generated when the object is irradiated with the light from the irradiating unit, at a position opposing the irradiating unit across the object, and output an acoustic wave signal;

a probe controlling unit configured to control the probe when the probe receives the acoustic wave;

a control processor configured to control at least one of the irradiation position controlling unit and the probe controlling unit such that the light does not enter the probe directly without going through the object; and

a constructing unit configured to construct characteristic information on an inside of the object from the acoustic wave signal.

According to the present invention, it becomes possible to prevent the generation of the photoacoustic wave caused by direct irradiation of light to the probe in the photoacoustic tomography.

Further features of the present invention will become apparent from the following description of exemplary embodiments with reference to the attached drawings.

### BRIEF DESCRIPTION OF THE DRAWINGS

FIG. 1 is a schematic view of a configuration of an object information acquiring apparatus in a first embodiment;

FIG. 2 is a flowchart of acquisition of object information in the first embodiment;

FIGS. 3A and 3B are conceptual views for explaining basic scanning control in the first embodiment;

FIGS. 4A to 4C are conceptual views for explaining selective scanning control in the first embodiment;

FIGS. 5A to 5F are conceptual views for explaining a time-series operation of the selective scanning control in the first embodiment;

FIGS. 6A to 6E are conceptual views for explaining the time-series operation of the selective scanning control in the first embodiment;

FIGS. 7A to 7D are conceptual views for explaining the time-series operation of the selective scanning control in the first embodiment;

FIG. 8 is a schematic view of a configuration of an object information acquiring apparatus in a second embodiment;

FIG. 9 is a flowchart of acquisition of object information in the second embodiment;

FIGS. 10A and 10B are conceptual views for explaining basic control of object information acquisition in the second embodiment;

FIGS. 11A and 11B are conceptual views for explaining acquisition control of the object information in the second embodiment;

FIGS. 12A and 12B are conceptual views for explaining the acquisition control of the object information in the second embodiment;

FIGS. 13A to 13D are conceptual views for explaining acquisition control of object information in a third embodiment;

FIGS. 14A to 14E are conceptual views for explaining a time-series operation of scanning control in the third embodiment;

FIGS. 15A to 15E are conceptual views for explaining the time-series operation of the scanning control in the third embodiment; and

FIGS. 16A and 16B are conceptual views for explaining the scanning control in the third embodiment.

### DESCRIPTION OF THE EMBODIMENTS

Hereinbelow, preferred embodiments of the present invention will be described with reference to the drawings. However, the dimension, material, shape, and relative arrangement of each component described below should be appropriately changed according to the configuration and various conditions of an apparatus to which the present invention is applied, and are not intended to limit the scope of the present invention to the following description.

In the present invention, an acoustic wave includes a sound wave, an ultrasonic wave, a photoacoustic wave, and an elastic wave called a photoultrasonic wave. That is, an object information acquiring apparatus of the present invention is an apparatus that receives an acoustic wave generated in an object due to a photoacoustic effect by irradiating the object with light (electromagnetic wave) to thereby acquire characteristic information on the inside of the object.

The characteristic information on the inside of the object acquired at this point includes object information that reflects an initial sound pressure of an acoustic wave generated by light irradiation, a light energy absorption density derived from the initial sound pressure, an absorption coefficient, and the concentration of a material constituting a tissue. An example of the concentration of the material includes an oxygen saturation, an oxyhemoglobin concentration, or a deoxyhemoglobin concentration. In addition, as the characteristic information, distribution information at each position in the object may be acquired instead of numerical data. That is, distribution information such as an absorption coefficient distribution or an oxygen saturation distribution may be acquired as image data.

Hereinbelow, the present invention will be described in detail with reference to the drawings. Note that the same components are denoted by the same reference numerals in principle and repeated description thereof will be omitted. The present invention can also be considered as an object information acquiring apparatus, an operation method thereof, and a control method thereof. The present invention can also be considered as a program for causing an information processing apparatus or the like to execute the control method.

### First Embodiment

The object information acquiring apparatus of the present invention has an opposing configuration in which an irradiation opening of pulsed light and a probe oppose each other across the object. The apparatus receives the photoacoustic wave generated from the object irradiated with the pulsed light, and generates a photoacoustic wave signal from the photoacoustic wave. In addition, the apparatus can acquire the object information in a wade region by performing two-dimensional scanning using the irradiation position of the pulsed light and reception position of the probe.

A feature of the present embodiment is that it is possible to excellently acquire the object information in the wide region by determining the scanning region of the pulsed light correspondingly to the shape of the object.

In addition, in the present embodiment, prior to the acquisition of the photoacoustic wave, the probe scan the object two-dimensionally while performing transmission and reception of an ultrasonic wave, and the object shape is thereby acquired in advance.

In order to acquire the object shape, the object information acquiring apparatus of the present embodiment can transmit the ultrasonic wave to the object and receive a reflected wave (ultrasonic echo). The inside of the object can be imaged by using two types of modalities based on the photoacoustic wave and the ultrasonic echo. The object information generated from the ultrasonic echo reflects a difference in the acoustic impedance of the tissue in the object.

The probe in the present embodiment receives the photoacoustic eave and the ultrasonic wave that are generated or reflected in the object. An electrical signal outputted by the probe after the probe receives the photoacoustic wave is referred to as a photoacoustic wave signal. In addition, an electrical signal outputted by the probe after the probe receives the ultrasonic echo is referred to as an ultrasonic wave signal. Each of the photoacoustic wave signal and the ultrasonic wave signal is a concept that includes an analog signal outputted from the probe, a signal subjected to amplification processing, and a signal subjected to digital conversion.

### (Component and Function)

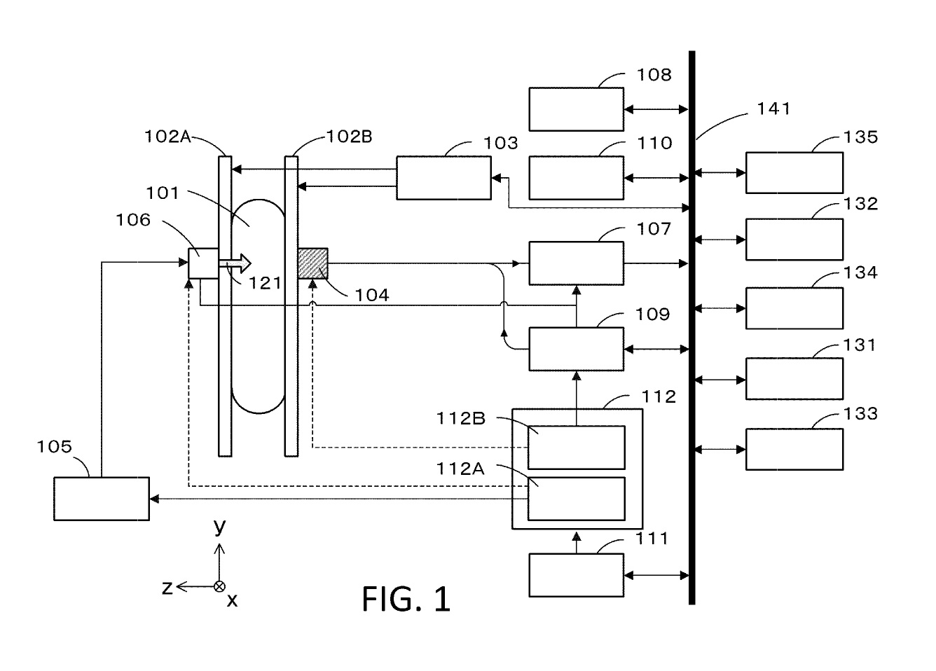

FIG. 1 is a schematic view of the configuration of the object information acquiring apparatus in the first embodiment.

The object information acquiring apparatus in the first embodiment includes a holding plate 102 that holds an object 101 and a holding control section 103 that maintains the holding in a state suitable for measurement. The apparatus also includes a probe 104 that performs reception of the photoacoustic wave and transmission and reception of the ultrasonic wave, a light source 105 that generates light, and an irradiation optical system 106 that irradiates the object 101 with light 121.

In addition, the apparatus also includes a signal reception section 107 that amplifies the electrical signal detected by the probe 104 and converts the electrical signal into a digital signal, and a photoacoustic wave signal processing section 108 that performs integration of the photoacoustic wave signal. Further, the apparatus includes an ultrasonic wave transmission control section 109 that applies an ultrasonic wave transmission drive signal to the probe 104, and an ultrasonic wave signal processing section 110 that performs reception focus processing on the ultrasonic wave signal. Furthermore, the apparatus includes an operation section 131 for inputting instructions for start of measurement and the like and parameters required for the measurement into the apparatus by a user (mainly an examiner such as medical staff or the like).

In addition, the apparatus also includes an image construction section 132 that constructs a photoacoustic wave image and an ultrasonic wave image from the photoacoustic wave signal and the ultrasonic wave signal, and a display section 133 that displays a user interface (UI) for operating the images and the apparatus. Further, the apparatus also includes a control processor 111 that receives various operations by the user via the operation section 131 and generates control information required for measurement operations. The control processor transmits the control information via a system bus 141 to thereby control the individual components of the apparatus. Furthermore, the apparatus also includes a position control section 112 that two-dimensionally controls the irradiation position of the light 121 and the position of the probe 104, and a storage section 134 that stores setting information related to the acquired signal or the measurement operations.

The object 101 serving as the measurement target is, e.g., a breast in breast cancer diagnosis in a breast oncology department. However, the object is not limited thereto. The apparatus of the present invention can measure various samples such as body tissues and phantoms.

The holding plate 102 is configured by a pair of holding plates 102A and 102B that are controlled by the holding control section 103 so as to have a holding distance therebetween as an interval suitable for the measurement. In the case where it is not necessary to differentiate between the holding plates 102A and 102B, they are collectively described as the holding plate 102. By pinching and fixing the object 101 using the holding plate 102, it is possible to reduce a measurement error caused by the movement of the object 101.

Note that the holding plate 102B positioned in a propagation path of the ultrasonic wave is preferably formed of a material having high acoustic matching with the probe 104. In addition, by using an acoustic matching material such as a gel sheet for ultrasonic wave measurement or the like, it is possible to enhance acoustic coupling between the probe 104 and the holding plate 102B or between the holding plate 102B and the object 101.

The holding control section 103 adjusts the holding state of the object 101 in accordance with the burden of a subject and the measurement depth as a target. The holding state includes the holding distance and a holding pressure, and has preferable values for each measurement of the photoacoustic wave or the ultrasonic wave.

The holding control section 103 also includes a lock mechanism of the holding state (not shown). The user can determine the holding state by turning on a switch of the lock mechanism to thereby fix the object. The holding control section 103 controls the holding state of the object 101 such that the holding state thereof is kept constant during the measurement except when a request from the subject or a holding release operation by the user is made. The holding control section 103 also outputs holding information indicative of the holding state (the holding distance and the holding pressure) of the object 101 to the control processor 111.

In the probe 104, a plurality of acoustic elements are arranged. The acoustic elements detect the photoacoustic wave generated in the object irradiated with the pulsed light 121, and convert the detected photoacoustic wave into the analog electrical signal. The probe for photoacoustic wave reception can also be used as the probe for ultrasonic wave reception. In this case, the acoustic elements transmit the ultrasonic wave to the object 101, detect an ultrasonic echo reflected in the object, and convert the detected ultrasonic echo into the analog electrical signal. The plurality of the acoustic elements are arranged along at least a first direction. If the first direction intersects the scanning direction of the probe, it is possible to measure the wide region of the object appropriately.

As long as the object of the present invention can be achieved, the system of the probe is not limited. For example, it is possible to use a transducer that uses piezoelectric ceramics (PZT). In addition, it is also possible to use a capacitive type capacitive micromachined ultrasonic transducer (CMUT) and a magnetic MUT (MMUT) that uses a magnetic film. Further, it is also possible to use a piezoelectric MUT (PMUT) that uses a piezoelectric thin film.

Note that the probe 104 is preferably capable of transmission of the ultrasonic wave and reception of the ultrasonic echo and the photoacoustic wave. With this, it is possible to acquire the object information derived from the ultrasonic wave and the object information derived from the photoacoustic wave at the same position, and reduce cost. However, the present invention can also be implemented by the apparatus that has the probe dedicated to the transmission and reception of the ultrasonic wave and the probe dedicated to the reception of the photoacoustic wave, and their respective signal reception systems.

As the probe 104, an array probe in which a plurality of the acoustic elements are arranged two-dimensionally is preferable. With this, it is possible to detect the photoacoustic wave that is three-dimensionally generated from a generation source such as a light absorbing material and propagates at the widest possible solid angle. As a result, it is possible to receive the photoacoustic wave and the ultrasonic wave required to excellently image the object area at the front of the probe.

The light source 105 emits pulsed light having a center wavelength in a near infrared range of 530 nm to 1300 nm. The pulse width of the pulsed light is preferably not more than 100 nsec. As the light source 105, in general, there is used a solid state laser capable of emitting the pulsed light having a center wavelength in the rear infrared range (e.g., Yttrium-Aluminum-Garnet laser or Titan-Sapphire laser). As the light source 105, lasers such as a gas laser, a dye laser, and a semiconductor laser can also be used. In addition, instead of the lasers, a light emitting diode can also be used.

Note that the wavelength of the light is selected according to the light absorbing material in the living body as the measurement target. For example, generally speaking, hemoglobin in a new blood vessel of breast cancer mainly absorbs light of 600 nm to 100 nm. On the other hand, light absorption of water constituting the living body becomes minimal in the vicinity of 830 nm. Accordingly, the light absorption in 750 nm to 850 nm becomes relatively large. In addition, the light absorption rate for each wavelength is changed according to the state of hemoglobin (oxygen saturation), and hence the functional change of the living body can be measured by utilizing this wavelength dependence.

The light source 105 includes a shutter for performing output control of the generated pulsed light and an optical configuration for controlling the wavelength of the pulsed light.

The irradiation optical system 106 guides the pulsed light emitted by the light source 105 to the object, and emits the light 121 suitable for the measurement from an emission end. The irradiation optical system 106 includes optical components such as a lens that condenses and magnifies the light, a prism, a mirror that reflects the light, a diffusion plate that diffuses the light, and an optical fiber that guides the light. The light source and the irradiation optical system correspond to an irradiating unit of the present invention.

Note that, as safety standards related to irradiation of the laser light to a skin and an eye, the maximum permissible exposure (MPE) having the wavelength of light, exposure duration, and pulse repetition as conditions is determined. The irradiation optical system 106 generates the light 121 having a shape and an emission angle suitable for imaging the object area at the front of the probe 104 after securing the safety for the object 101.

In addition, the irradiation optical system 106 includes an optical configuration (not shown) that detects the emission of the light 121 to the object 101, and generates a synchronization signal for controlling reception and record of the photoacoustic wave in synchronization with the detection. In order to detect the emission of the light 121, a part of the pulsed light generated by the light source 105 is divided using a half mirror or the like, and the light obtained by the division is guided to an optical sensor in advance. Subsequently, the optical configuration (not shown) monitors a detection signal outputted from the optical sensor and generates the synchronization signal. In the case where a bundle fiber is used to guide the pulsed light, a part of the fiber may be branched and the pulsed light may be guided to the optical sensor. The generated synchronization signal is inputted to the signal reception section 107.

The signal reception section 107 includes a signal amplification section that amplifies the analog signal generated by the probe 104, and an A/D conversion section that converts the analog signal into the digital signal. The signal reception section 107 performs amplification processing and digital conversion on the analog photoacoustic wave signal or ultrasonic wave signal generated by the probe 104 in synchronization with the synchronization signal sent from the irradiation optical system 106 or the ultrasonic wave transmission control section 109.

The photoacoustic wave signal processing section 108 performs various processing on the digital signal outputted from the signal reception section 107. Examples of the processing include sensitivity variation correction of the acoustic element of the probe 104, complementary processing of the physically or electrically damaged element, and integration processing for noise reduction.

The photoacoustic wave signal processing section 108 also has the function of integrating a plurality of the photoacoustic wave signals at the same position obtained by the two-dimensional scanning of the probe 104 in the form of a two-dimensional array. With this, effects such as an improvement in S/N ratio and signal complementing are obtained.

The ultrasonic wave transmission control section 109 generates drive signals applied to the individual acoustic elements constituting the probe 104 and controls the frequency and the sound pressure of the ultrasonic wave to be transmitted. In the first embodiment, the array probe in which a plurality of the acoustic elements are arranged two-dimensionally is used. The ultrasonic wave transmission control section 109 performs linear scanning of transmission of an ultrasonic beam and reception of the ultrasonic echo along one direction constituting the array. By repeatedly performing the linear scanning along scanning of the probe 104, three-dimensional ultrasonic wave signal data configured by a plurality of B-mode images is obtained.

Transmission control is performed by setting the transmission direction of the ultrasonic beam and selecting a transmission delay pattern correspondingly to the transmission direction. On the other hand, reception control is performed by setting the reception direction of the ultrasonic echo and selecting a reception delay pattern correspondingly to the reception direction.

Note that a description is given herein on the assumption that the ultrasonic wave transmission control section 109 has the transmission control function and the reception control function of the ultrasonic wave, but another component may be caused to perform the reception control.

The transmission delay pattern is a pattern of delay time given to a plurality of the drive signals in order to form the ultrasonic beam in a predetermined direction using the ultrasonic wave transmitted from the plurality of the acoustic elements. The reception delay pattern is a pattern of delay time given to a plurality of the reception signals in order to extract the ultrasonic echo from any direction relative to the ultrasonic wave signal detected by the plurality of the acoustic elements. The transmission delay pattern and the reception delay pattern are stored in the storage section 134.

The ultrasonic wave signal processing section 110 performs reception focus processing based on the selected reception delay pattern. Specifically, the ultrasonic wave signal processing section 110 performs delay processing corresponding to the delay time on each of the ultrasonic wave signals generated by the signal reception section 107, and then integrates the individual signals. With this processing, focused ultrasonic wave signal data is generated. The ultrasonic wave signal processing section 110 may further perform logarithmic compression or filtering. In this manner, the B-mode image is generated.

The control processor 111 operates an operation system (OS) that performs control and management of basic resources in program operations. The control processor 111 also read a program code stored in the storage section 134 and executes each processing of the embodiments described later.

The control processor 111 especially receives event notifications generated by various operations such as an instruction to start or suspend the acquisition of the object information from the user via the operation section 131, and manages acquisition operations of the object information. At this point, the control processor 111 controls each hardware via the system bus 141. The system bus 141 is assumed to include a general-purpose expansion bus for connecting peripheral equipment such as PCI express or USB.

The control processor 111 also generates scanning control information related to the acquisition position or the acquisition region of the object information based on a parameter specified from the operation section 131 or a parameter pre-set in the storage section 134, and outputs the scanning control information to the position control section 112. In the present embodiment, the control processor 111 generates the scanning control information corresponding to the object shape acquired by the shape acquisition section 135, and outputs the scanning control information to the position control section 112.

The control processor 111 outputs output control information of the pulsed light 121 required for the reception operation of the photoacoustic wave signal to a light irradiation position control section 112A. The control processor 111 outputs control information related to the ultrasonic wave transmission/reception control operations such as setting of a plurality of focuses and the like to the ultrasonic wave transmission control section 109 and the ultrasonic wave signal processing section 110.

The position control section 112 includes the light irradiation position control section 112A and a probe position control section 112B. The light irradiation position control section 112A controls the irradiation position of the light 121 on the holding plate 102A according to the scanning control information from the control processor 111. The probe position control section 112B controls the position of the probe 104 on the holding plate 102B. Note that, in the case where it is not necessary to differentiate between them, they are simply referred to as the position control section 112. The light irradiation position control section corresponds to an irradiation position controlling unit of the present invention. The probe position control section corresponds to a probe controlling unit of the present invention.

The light irradiation position control section 112A and the probe position control section 112B control a light irradiation position from the emission end and a probe position using movement mechanisms (not shown) of them. The movement mechanisms are configured by drive members such as motors or the like and mechanical components for transmitting driving forces thereof, and can individually control the position of the light 121 and the position of the probe 104. By repeating the measurement while performing the two-dimensional scanning using the irradiation position of the light 121 and the position of the probe 104 on the object 101, it becomes possible to measure a wide region even with a small probe.

The position control section 112 moves the light irradiation position and the probe position to points required to generate the target object information in synchronization with a light emission repetition period of the pulsed light 121 by the light source 105. The light irradiation position control section 112A further instructs the light source 105 to perform opening/closing control of the shutter such that the object 101 is irradiated with the pulsed light 121 the number of times required to acquire the photoacoustic wave signal during continuous movement control.

The position control section 112 further outputs coordinate information on each of the irradiation position of the light 121 and the position of the probe 104 to the control processor 111 each time the light irradiation is performed (or each time the photoacoustic wave signal corresponding to one light irradiation is acquired). Thus, by retaining the coordinate information on each of the light irradiation position and the probe position stored every time the photoacoustic wave signal is acquired, it is possible to accurately execute image reconstruction processing.

In addition, in the case where the object information is acquired by using the ultrasonic echo as in the present embodiment, the probe position control section 112B instructs the ultrasonic wave transmission control section 109 to start the linear scanning of the ultrasonic beam.

Note that, although the position control section 112 is described as an independent configuration in the present embodiment, the individual functions of the position control section 112 may be executed by the control processor 111.

The operation section 131 is an input apparatus for specifying a parameter related to the acquisition operation of the object information by the user. The parameter includes a measurement position and a measurement region. In addition, reception gain may be set for each of the photoacoustic wave and the ultrasonic wave. The operation section 131 is configured by, e.g., a mouse, a keyboard, or a touch panel, and performs the event notification to software such as OS operating on the control processor 111 according to the operation of the user.

The image construction section 132 generates a tomographic image representing a photoacoustic wave image or an ultrasonic wave image of the inside of the object, or a display image in which the images are superimposed on each other. The image construction section 132 can also apply various correction processing such as brightness correction, distortion correction, and trimming of a target area to the generated image. The image construction section 132 also performs adjustment of parameters related to the construction of the photoacoustic wave image, the ultrasonic wave image, or the superimposition image thereof, and the display image according to the operation of the operation section 131 by the user. The image construction section corresponds to a constructing unit of the present invention.

The photoacoustic wave image is an image in which the object information such as an optical characteristic value distribution or the like and the functional information such as the oxygen saturation or the like are visualized. On the other hand, the ultrasonic wave image shows a change in acoustic impedance in the object.

As the image reconstruction processing, there is used, e.g., back projection in a time domain or Fourier domain or phasing addition processing that are commonly used in tomography technologies. Note that, in the case where time constraints are not severe, it is possible to use an image reconstruction method such as an inverse problem analysis method using iteration. By using the probe having a reception focus function with an acoustic lens, it is also possible to visualize the object information without performing the image reconstruction.

In the image construction section 132, a graphics processing unit (GPU) having a high arithmetic processing function and a graphic display function or the like is commonly used.

The display section 133 displays the photoacoustic wave image and the ultrasonic wave image constructed by the image construction section 132 or the superimposition image thereof, and a UI for operating the images and apparatus. The display section 133 may be a display of any system such as a liquid crystal display or an organic EL display.

The storage section 134 stores and retains information required for the operation of the control processor 111, temporary data, the generated photoacoustic wave image and ultrasonic wave image, relevant object information, and diagnosis information. The storage section 134 also stores the program code of software that implements the functions of each embodiment. The storage section 134 is configured by a storage medium such as a hard disk or a nonvolatile memory.

The shape acquisition section 135 generates shape information on the held object 101 based on signal data of the ultrasonic wave acquired in a large region within which the entire object 101 can fall in advance. The generation of the shape information may be performed by using, e.g., existing shape recognition technologies. Note that, although the shape acquisition section 135 is described as an independent configuration in the present embodiment, the control processor 111 may be caused to execute the function of the shape acquisition section 135.

According to the object information acquiring apparatus having the above-described configuration, it is possible to measure the object information while the irradiation position of the light 121 and the position of the probe 104 are controlled independently of each other. In addition, it is also possible to acquire the photoacoustic wave image and the ultrasonic wave image of the same object area.

### (Processing Flow)

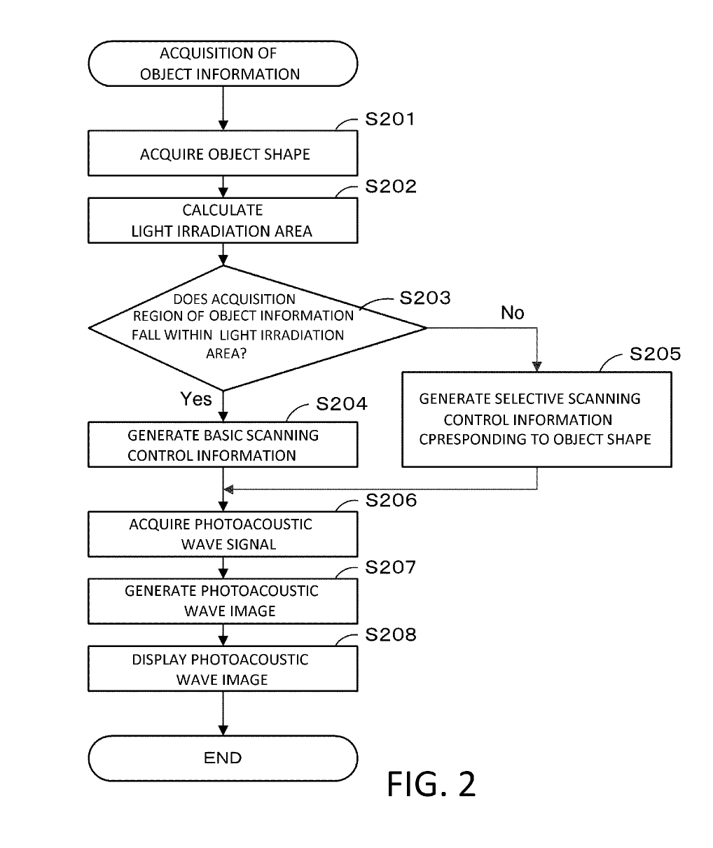

With reference to a flowchart of FIG. 2, the flow of acquisition of the object information in the first embodiment will be described. The flow of FIG. 2 is started when the user sets the acquisition region of the object information and the parameter required to generate the target object information via the operation section 131 and issues an instruction to start the acquisition of the object information.

In Step S201, prior to the acquisition of the object information using the photoacoustic wave, the control processor 111 instructs the probe position control section 112B to acquire the shape of the object 101 by using the ultrasonic wave. The probe position control section 112B controls the ultrasonic wave transmission control section 109 to acquire three-dimensional ultrasonic wave signal data including the shape of the project 101.

Note that the purpose of the two-dimensional scanning in this step is to acquire the shape of the object 101, especially outline information, and hence high resolution is not required. Instead, it is preferable to execute rough linear scanning suitable for acquisition of only the target shape information in a short time period. Accordingly, the maximum acquisition region of the object information determined as the specifications of the apparatus is two-dimensionally scanned.

Subsequently, the control processor 111 obtains information on the object shape based on the acquired ultrasonic wave signal data from the shape acquisition section 135.

In Step S202, the control processor 111 refers to the shape information on the object 101 acquired in Step S201 and the shape of the light 121 to calculate coordinate information that allows the shape of the light 121 to fall within the region of the object shape. The coordinate information represents a light irradiation area.

In Step S203, the control processor 111 determines whether or not the acquisition region of the object information specified by the user falls within the light irradiation area calculated in Step S202. The processing moves to Step S204 in the case where the acquisition region of the object information is within the light irradiation area (Yes), and the processing moves to Step S205 in the case where the acquisition region thereof does not fall within the light irradiation area (No).

In Step S204, the control processor 111 generates basic scanning control information related to two-dimensional scanning as the base, and outputs the basic scanning control information to the position control section 112.

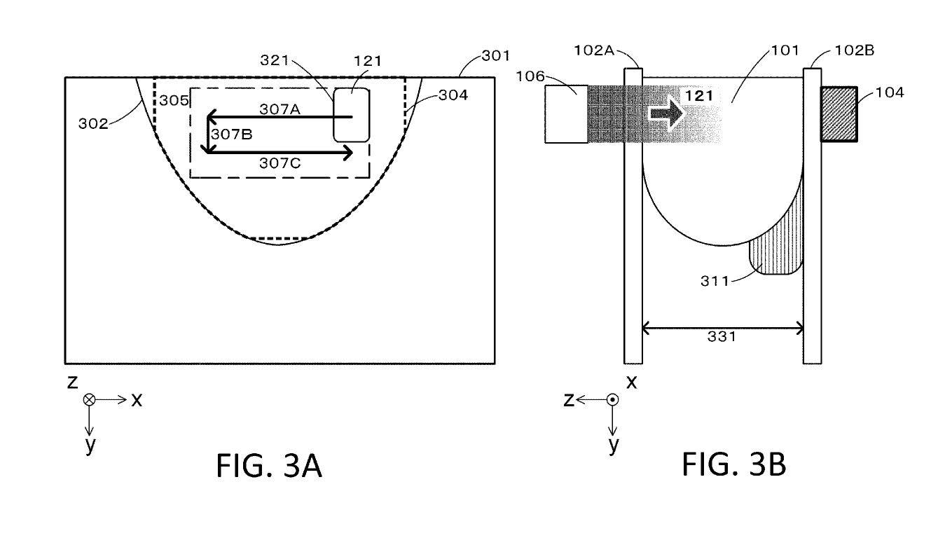

FIG. 3 is a conceptual view for explaining basic scanning control in the first embodiment. FIG. 3A is a front view of the held object 101 as viewed from the side of the holding plate 102A. FIG. 3B is a side view thereof.

The reference numeral 301 indicates the maximum region in which the object information can be acquired as the specifications of the apparatus. The reference numeral 302 indicates a line indicative of the shape of the object 101 held at a holding distance 331 that is acquired in Step S201, i.e., an outermost outline.

The reference numeral 321 indicates the shape of the pulsed light 121 on an xy plane. In the first embodiment, the pulsed light shape 321 is smaller than the object 101, and has a distribution shape corresponding to the size of the probe 104.

The reference numeral 304 indicates the light irradiation area calculated in Step S202 in which the light shape 321 falls within the region of the object shape 302. All of the pulsed light 121 emitted in the light irradiation area 304 enters the object 101, and hence a part or all of the pulsed light 121 does not reach the side of the holding plate 102B. Consequently, at least a part of the pulsed light 121 goes through the object, and the pulsed light 121 having high energy is prevented from entering the probe 104.

Note that, in FIG. 3, the light irradiation area 304 is set along the outermost outline 302 of the object 101. However, some margin may be provided in consideration of the distribution shape of the pulsed light 121 corresponding to directivity, and a region slightly inside the reference numeral 304 may also be set as the light irradiation area 304. In the case where the object 101 is slightly displaced during the acquisition of the object information, it is possible to prevent a part of the pulsed light 121 from reaching the probe 104 while maintaining high energy.

Note that, in the first embodiment, for the sake of the description, it is assumed that the pulsed light 121 is collimated light having an ideal parallel characteristic, and is emitted at an angle orthogonal to a two-dimensional planar boundary surface between the holding plate 102A and the object 101.

However, the above method that provides the margin in the light irradiation area 304 is effective for the case where the pulsed light 121 is not complete coherent light or the case where diffraction caused by scattering may occur. In addition, even in the case where the pulsed light is not coherent light, the light irradiation position control section grasps the characteristic of directivity of light, and may appropriately control the irradiation position such that the irradiation light does not enter the probe directly.

The reference numeral 311 indicates the acoustic matching material such as an ultrasonic gel sheet for securing acoustic coupling by being filled in a gap between the object 101 and the holding plate 102B.

The reference numeral 305 indicates the acquisition region of the object information specified by the user in advance, and FIG. 3 shows the case where the entire acquisition range falls within the light irradiation area 304.

The reference numerals 307A, 307B, and 307C indicate scanning lines of the two-dimensional scanning required to acquire the photoacoustic wave from the region 305. The control processor 111 generates the scanning control information required to perform the two-dimensional scanning in the order of 307A, 307B, and 307C. Note that the scanning control information includes start and end positions of the scanning, a movement speed in the scanning line joining the two positions, information on acceleration before the movement speed is reached, and information on deceleration before stop of the scanning.

The control processor 111 passes the generated scanning control information to the position control section 112 in the next step, and entrusts the control of the two-dimensional scanning to the position control section 112.

It is assumed that, as shown in FIG. 3B, the position control section 112 basically performs two-dimensional scanning control while maintaining the opposing positional relationship between the irradiation position of the light 121 (i.e., the position of the irradiation optical system 106) and the probe 104. By maintaining the opposing positional relationship, it is possible to concentrate the energy of the pulsed light 121 on the area of the object 101 positioned at the front of the probe 104, and hence it is possible to acquire the photoacoustic wave with high energy efficiency.

Returning to FIG. 2, the description will be continued. In Step S205, the control processor 111 generates selective control information of the two-dimensional scanning corresponding to the object shape as the feature of the present invention, and outputs the selective control information to the position control section 112.

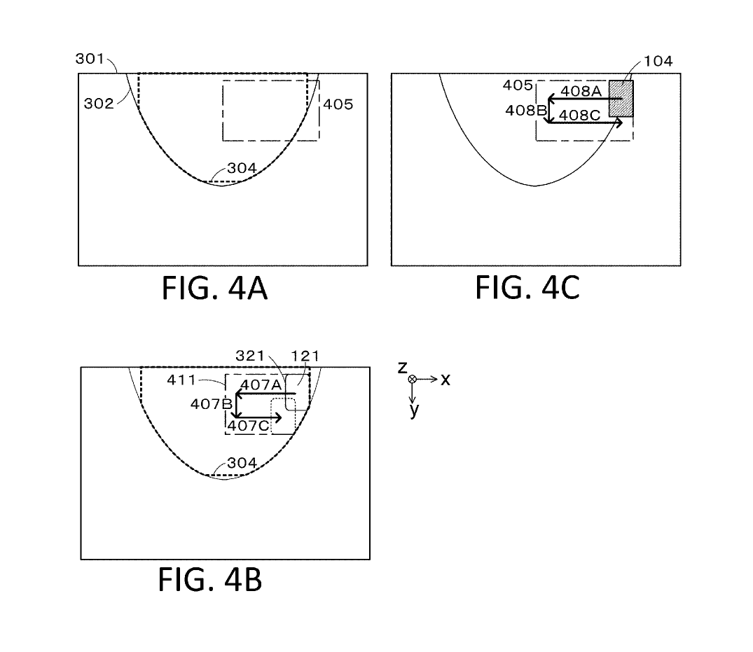

FIG. 4 is a conceptual view for explaining selective scanning control in the first embodiment. Similarly to FIG. 3A, each of FIGS. 4A to 4C is a front view of the held object 101 as viewed from the side of the holding plate 102A. For the sake of the description, in FIG. 4C, the probe 104 positioned on the depth side of the object 101 is projected on the drawing.

FIG. 4A shows that an acquisition region 405 of the object information specified by the user does not fall within the light irradiation area 304. When the basic scanning control shown in FIG. 3 is performed on the region 405, in the peripheral edge part of the object 101, a part of the pulsed light 121 reaches the probe 104 while maintaining high energy without entering the object 101. Thus, as the result of the entry of at least a part of the pulsed light 121 into the probe 104 without going through the object, a strong signal of the photoacoustic wave generated by the surface of the probe or a reflective film becomes manifest as a noise in the photoacoustic image.

To cope with this, in the first embodiment, in the case where the region 405 shown in FIG. 4A is specified, the scanning control of the light irradiation position shown in FIG. 4B is performed correspondingly to the object shape. With this, the pulsed light 121 is selectively emitted to the object 101, and hence the pulsed light 121 does not reach the probe 104.

The control processor 111 generates the scanning control information required to perform the scanning using the light irradiation position in the order of scanning lines 407A, 407B, and 407C based on an overlap region 411 between the specified region 405 and the light irradiation area 304. Subsequently, in the next step, the control processor 111 passes the selective scanning control information to the light irradiation position control section 112A, and entrusts the control of the two-dimensional scanning to the light irradiation position control section 112A.

On the other hand, for the two-dimensional scanning of the probe 104, the control processor 111 generates the scanning control information required to perform the scanning using the probe position in the order of scanning lines 408A, 408B and 408C in order to acquire the photoacoustic wave from the specified region 405. Subsequently, in the next step, the control processor 111 passes the scanning control information to the probe position control section 112B, and entrusts the control of the two-dimensional scanning to the probe position control section 112B.

In Step S206, the control processor 111 passes the basic scanning control information generated in Step S204 or the selective scanning control information generated in Step S205 to the position control section 112 and causes the position control section 112 to start the acquisition operation of the object information. The position control section 112 acquires the photoacoustic wave required to generate the target object information and performs the scanning required to generate the photoacoustic wave signal according to the passed scanning control information.

In Step S207, the image construction section 132 performs image reconstruction processing on the photoacoustic wave signal obtained as the result of Step S206. In addition, the image construction section 132 performs various correction processing and trimming processing on the reconstructed image on an as needed basis to thereby visualize the target object information.

In Step S208, the display section 133 displays the visualized object information.

### (Time-Series Operation of Selective Scanning Control)

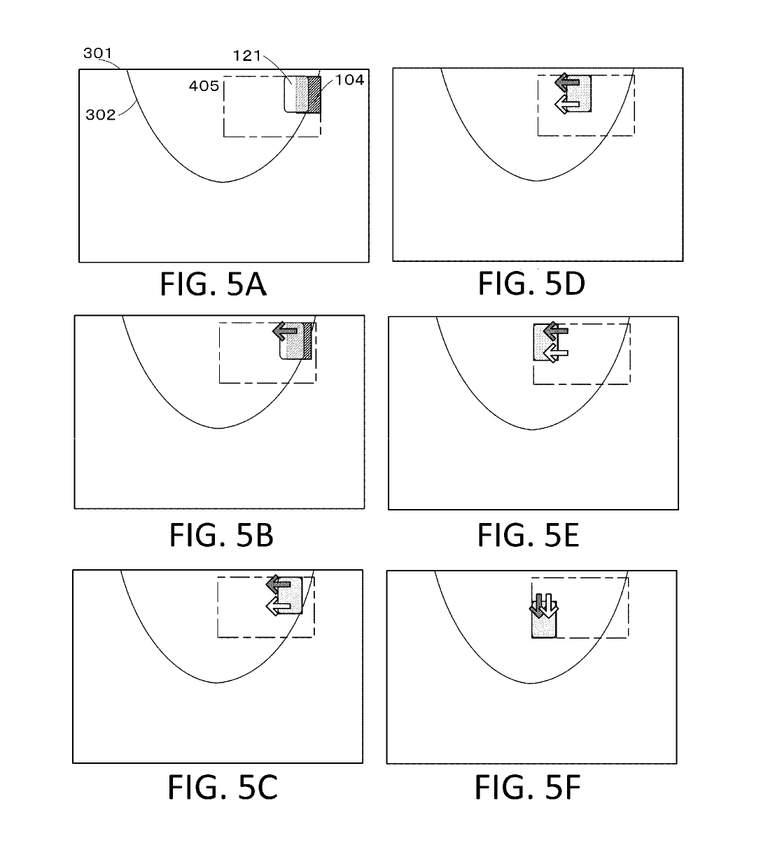

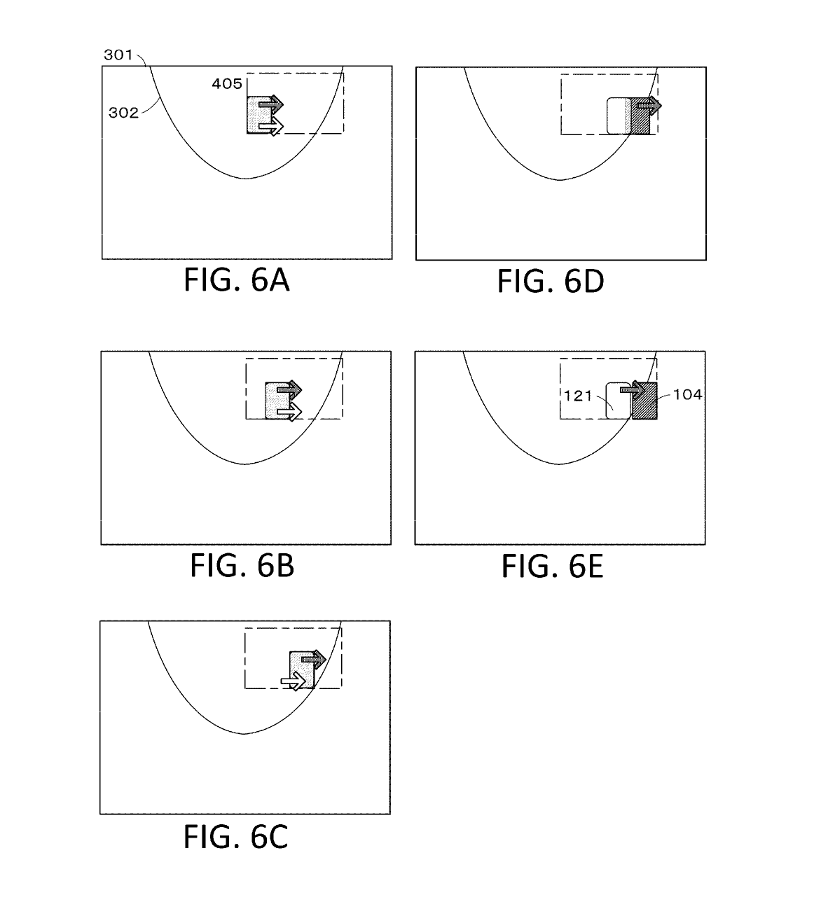

Subsequently, the time-series operation of the selective scanning control in the first embodiment will be described by using FIGS. 5 and 6.

FIGS. 5A to 5F show time-series operations of the irradiation position of the pulsed light 121 and the probe 104 from main scanning in a forward direction to sub-scanning performed thereafter in the specified acquisition region 405 of the object information. Note that a white arrow in the drawings is used to explain the movement of the light, and a gray arrow is used to explain the movement of the probe.

As shown in FIGS. 4B and 4C, the pulsed light 121 and the probe 104 have different contents of the two-dimensional scanning because the light irradiation to the object 101 is selectively controlled. The light irradiation position control section 112A controls the irradiation position of the pulsed light 121 in the order of the scanning lines 407A, 407B, and 407C according to the scanning control information. On the other hand, the probe position control section 112B controls the position of the probe 104 in the order of the scanning lines 408A, 408B, and 408C. Accordingly, the light 121 and the probe 104 have different time-series operations.

FIG. 5A shows a state in which the irradiation position of the light 121 and the position of the probe 104 have moved from wait positions (not shown) before the acquisition start to scanning start positions in synchronization with the acquisition start of the object information.

Thereafter, as shown in FIG. 5B, the probe position control section 112B moves the probe 104 along the scanning line 408A first, and the main scanning is thereby started. During this operation, the light irradiation position control section 112A keeps the irradiation position of the light 121 at the same position and repeats the irradiation of the pulsed light 121 to the object 101. The probe performs the acquisition of the photoacoustic wave for each light irradiation. According to this control, all of the pulsed light 121 enters the object 101. That is, a part or all of the pulsed light 121 is prevented from reaching the probe 104 while maintaining high energy, and it is possible to acquire the photoacoustic wave from the peripheral edge part of the object 101.

Note that the light irradiation position control section 112A does not start the scanning control of the light 121 until the probe 104 reaches the position of the light 121.

Next, as shown in FIG. 5C, the light irradiation position control section 112A starts the scanning using the irradiation position of the light 121 along the scanning line 407A in synchronization with the arrival of the probe 104 at the scanning start position of the light 121. After both of them overlap each other, the light irradiation position control section 112A and the probe position control section 112B performs the same scanning control on the irradiation position of the light 121 and the probe 104.

In FIG. 5D, the position control section 112 continues the main scanning while maintaining the opposing positional relationship between the pulsed light 121 and the probe 104, and repeats the acquisition of the photoacoustic wave required to generate the target object information.

As shown in FIG. 5E, when the main scanning of the uppermost line of the specified region 405 is completed, the position control section 112 decelerates and stops the scanning of each of the pulsed light 121 and the probe 104.

In FIG. 5F, the position control section 112 performs the scanning control of the position of the pulsed light 121 and the position of the probe 104 in a sub-scanning direction.

FIGS. 6A to 6E show the time-series operations in the main scanning in a backward direction in the specified acquisition region 405 of the object information.

In FIG. 6A, the position control section 112 starts the main scanning using the irradiation position of the pulsed light 121 and the position of the probe 104 in the backward direction. The scanning is performed along the scanning line 407C and the scanning line 408C.

In FIG. 6B, the position control section 112 continues the main scanning while maintaining the opposing positional relationship between the pulsed light 121 and the probe 104, and repeats the acquisition of the photoacoustic wave required to generate the target object information.

In FIG. 6C, since the scanning end position of the scanning line 407C is reached, the light irradiation position control section 112A decelerates and stops the scanning using the irradiation position of the pulsed light 121. Further, the light irradiation position control section 112A repeats the irradiation of the pulsed light 121 to the object 101 while keeping the irradiation position of the light 121 at the same position. On the other hand, the probe position control section 112B continues the main scanning along the scanning line 408C, and repeats the acquisition of the photoacoustic wave required to generate the target object information.

In FIG. 6D, the probe position control section 112B decelerates and stops the scanning of the probe 104 in order to complete the main scanning of the specified region 405.

Thereafter, for the next acquisition of the object information, the position control section 112 moves the irradiation position of the light 121 and the position of the probe 104 to the wait positions as shown in FIG. 6E.

With the above-described operations, the selective operation control related to the acquisition of the photoacoustic wave required to generate the target object information is completed.

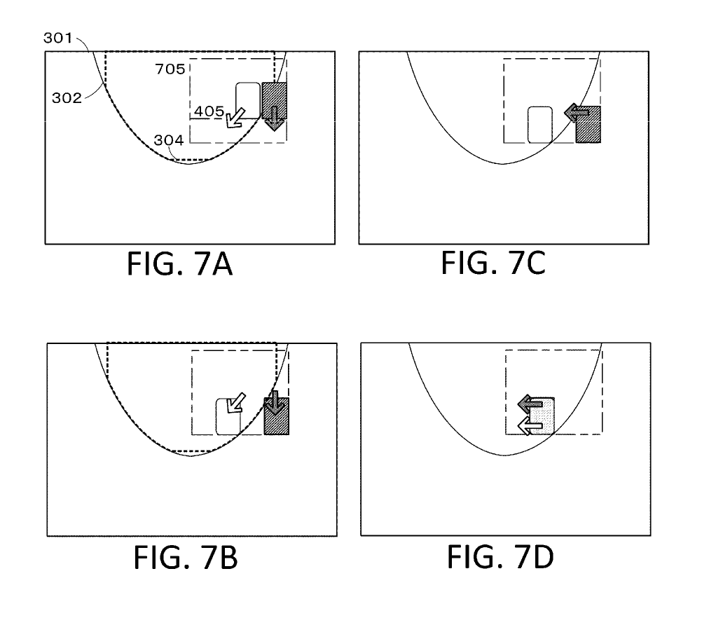

FIG. 7 is a conceptual view for explaining the time-series operation of the selective scanning control after FIG. 6E in the case where an acquisition region 705 of the object information that is wider than the region 405 in a y-axis direction is specified by the user.

In FIG. 7A, the scanning control in the sub-scanning direction is performed from the irradiation position of the light 121 and the position of the probe 104 shown in FIG. 6E toward the next main scanning start position. Note that the sub-scanning of the light 121 becomes a movement in an oblique direction indicated by a white arrow of FIG. 7A in order to set the scanning start position of the light 121 in the next main scanning to a position within the light irradiation area 304.

As the result of the sub-scanning shown in FIG. 7A, as shown in FIG. 7B, the irradiation position of the light 121 and the position of the probe 104 reach the start positions of the next main scanning in the forward direction.

In FIG. 7C, the probe position control section 112B moves the position of the probe 104 along the scanning line first to thereby start the main scanning. During this operation, the light irradiation position control section 112A keeps the irradiation position of the light 121 at the same position. During this operation as well, the light irradiation position control section 112A repeats the irradiation of the pulsed light 121 to the object 101. The probe 104 repeats the acquisition of the photoacoustic wave.

In FIG. 7D, the probe 104 reaches the scanning start position of the pulsed light 121. After both of them overlap each other, the light irradiation position control section 112A executes the scanning using the irradiation position of the pulsed light 121 along the scanning line 407A.

The main scanning is continued while the opposing positional relationship between the pulsed light 121 and the probe 104 is maintained, and the acquisition operation of the photoacoustic wave required to generate the target object information is completed.

With the object information acquiring method described above, it becomes possible to selectively control the irradiation position of the pulsed light 121 and its scanning to the object 101. As a result, the light is not directly emitted to the probe 104, and it is possible to prevent the generation of the strong photoacoustic wave on the surface of the probe 104 that becomes manifest as the noise when the object information is visualized.

### Second Embodiment

A second embodiment that uses the object information acquiring method of the present invention will be described according to the drawings.

In the first embodiment, in the configuration in which the target object information in the wide region is acquired by the mechanical two-dimensional scanning using the irradiation position of the pulsed light and the reception position of the probe, the selective scanning control of the light irradiation position is performed correspondingly to the object shape.

In the present embodiment, the acquisition of the object information in the object area positioned at the front of the probe is performed at the specified position without performing the two-dimensional scanning. Hereinbelow, characteristic parts of the present embodiment will be mainly described.

In the object information acquiring method of the present embodiment, when the object shape is acquired prior to the acquisition of the photoacoustic wave, instead of performing the two-dimensional scanning using the ultrasonic wave as in the first embodiment, the object is imaged using an imaging unit, and the image is analyzed.

### (Component and Function)

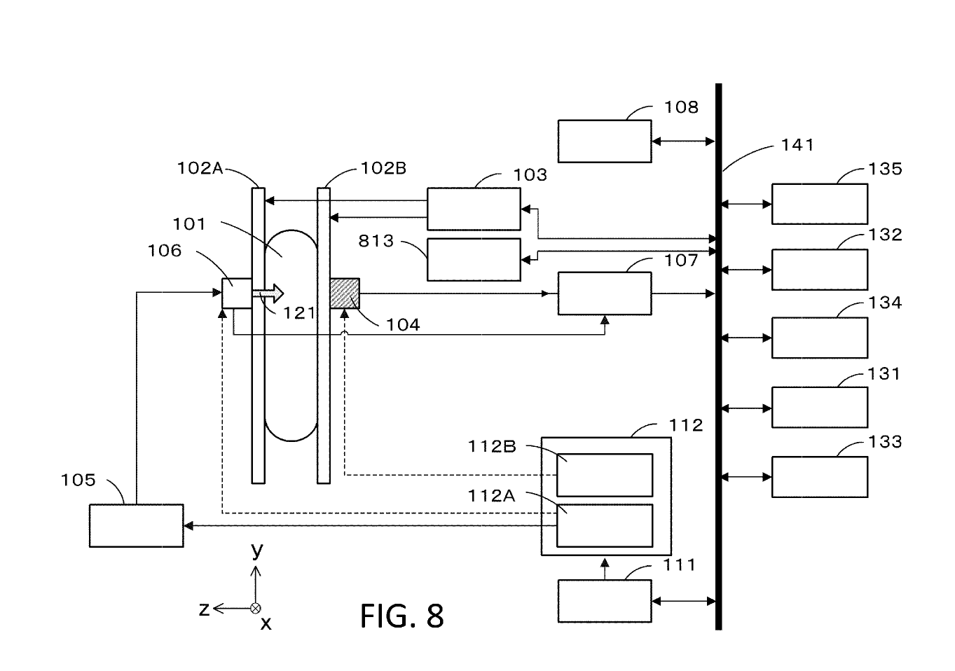

FIG. 8 is a schematic view of the configuration of an object information acquiring apparatus in the second embodiment.

A holding and imaging section 813 images the held object 101 via the holding plate 102A or 102B according to the instruction of the control processor 111. As an image sensor of the holding and imaging section 813, there can be used a common image sensor such as a CCD or CMOS image sensor having detection sensitivity in a visible region or an infrared region.

The image imaged by the holding and imaging section 813 is displayed on the display section 133, and is used for the specification of the measurement region by the user. Consequently, the holding and imaging section 813 preferably acquires the image of the entire maximum region determined as the specifications of the apparatus in which the object information can be acquired. For acquiring such an image, it is possible to use a method that widens the angle of view at the time of imaging and a method that synthesizes a plurality of images obtained by performing the imaging a plurality of times.

The image imaged by the holding and imaging section 813 is also used for acquiring information on the shape of the held object 101.

Note that, in FIG. 8, although the holding and imaging section 813 performs the imaging from the side of the probe 104 via the holding plate 102B, the imaging direction is not limited thereto. As long as the shape of the object 101 can be imaged, the holding and imaging section 813 may perform the imaging from the side of the irradiation optical system 106 via the holding plate 102A.

The shape acquisition section 135 generates the shape information on the held object 101 based on the image of the object 101 acquired by the holding and imaging section 813. The generation of the shape information may be performed by using, e.g., existing shape recognition technologies and image processing technologies such as skin color extraction and the like.

Note that the configurations and functions of the components other than the holding and imaging section and the shape acquisition section are the same as those described in FIG. 1 in the first embodiment.

### (Processing Flow)

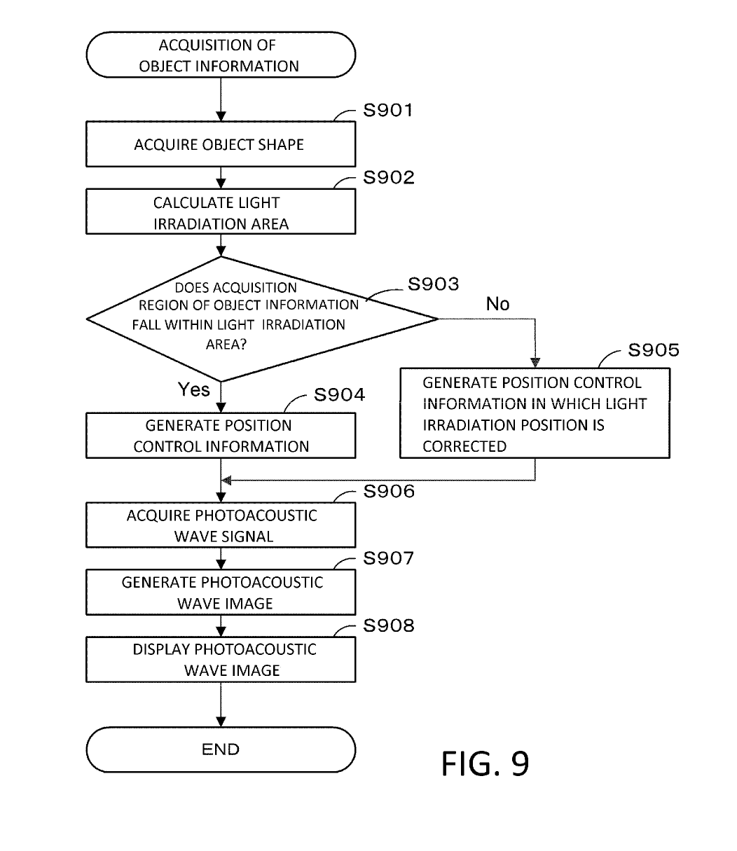

With reference to FIG. 9, the flowchart showing the flow of acquisition of the object information in the second embodiment will be described. The flowchart of FIG. 9 is executed when the user sets the acquisition region of the object information and the parameter required to generate the target object information via the operation section 131 and issues an instruction to start the acquisition of the object information.

In Step S901, the control processor 111 obtains the information on the object shape extracted by the shape acquisition section 135 based on the image of the object 101 imaged by the holding and imaging section 813.

In Step S902, similarly to S202 of FIG. 2, the control processor 111 refers to the shape information on the object 101 acquired in Step S901 and the shape of the light 121 to calculate the coordinate information that allows the shape of the light 121 to fall within the region of the object shape. The coordinate information represents the light irradiation area.

In Step S903, similarly to S203 of FIG. 2, the control processor 111 determines whether or not the acquisition position of the object information specified by the user falls within the light irradiation area calculated in Step S202. The processing moves to Step S904 in the case where the acquisition position of the object information is within the light irradiation area, and the processing moves to Step S905 in the case where the acquisition position thereof does not fall within the light irradiation area.

In Step S904, the control processor 111 generates position control information based on the acquisition position of the object information specified by the user.

In Step S905, the control processor 111 corrects the light irradiation position correspondingly to the object shape, and generates the position control information based on the corrected position.

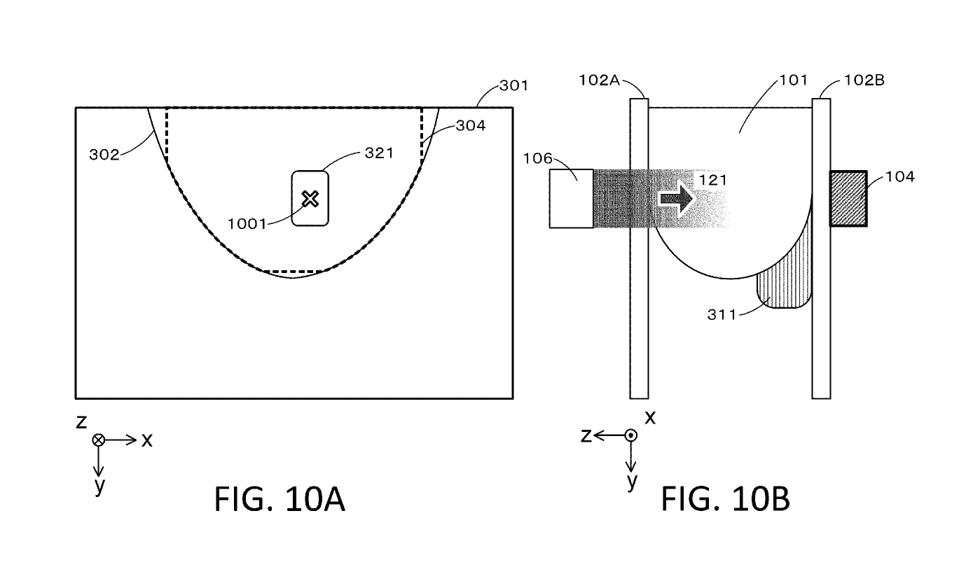

FIG. 10 is a conceptual view for explaining the basic control of the object information acquisition performed in the case where the acquisition position of the object information is within the light irradiation area in Step S903. FIG. 10A is a front view of the held object 101 as viewed from the side of the holding plate 102A. FIG. 10B is a side view thereof.

The reference numeral 1001 indicates the acquisition position of the object information specified by the user. The control processor 111 generates the position control information such that the center of the shape 321 of the pulsed light 121 and the center of the probe 104 match the acquisition position 1001. The position control section 112 acquires the photoacoustic wave according to the control information, and performs the scanning required to generate the photoacoustic wave signal.

At this position, as shown in FIG. 9B, all of the energy of the pulsed light 121 enters the object 101, and hence the pulsed light 121 does not reach the probe 104 while maintaining high energy.

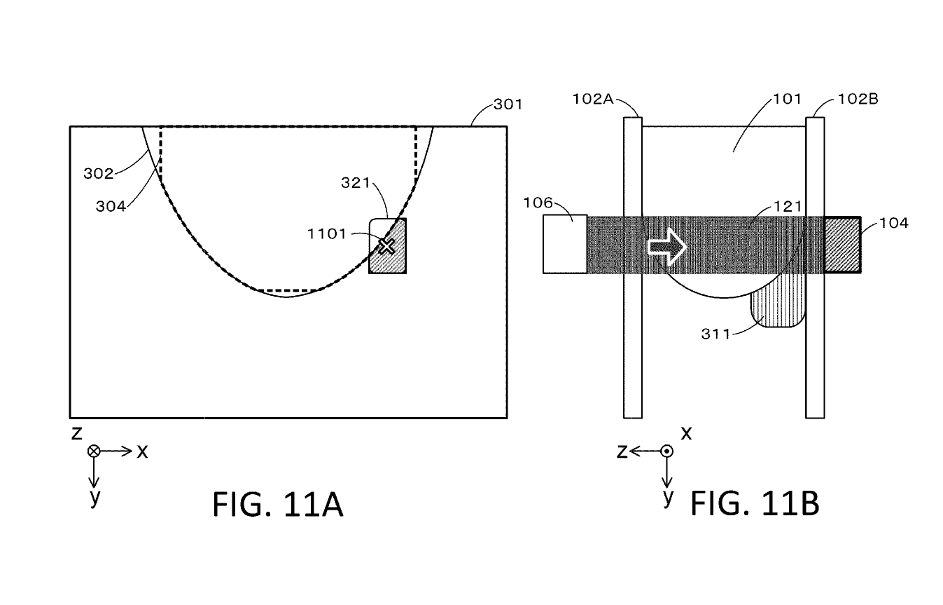

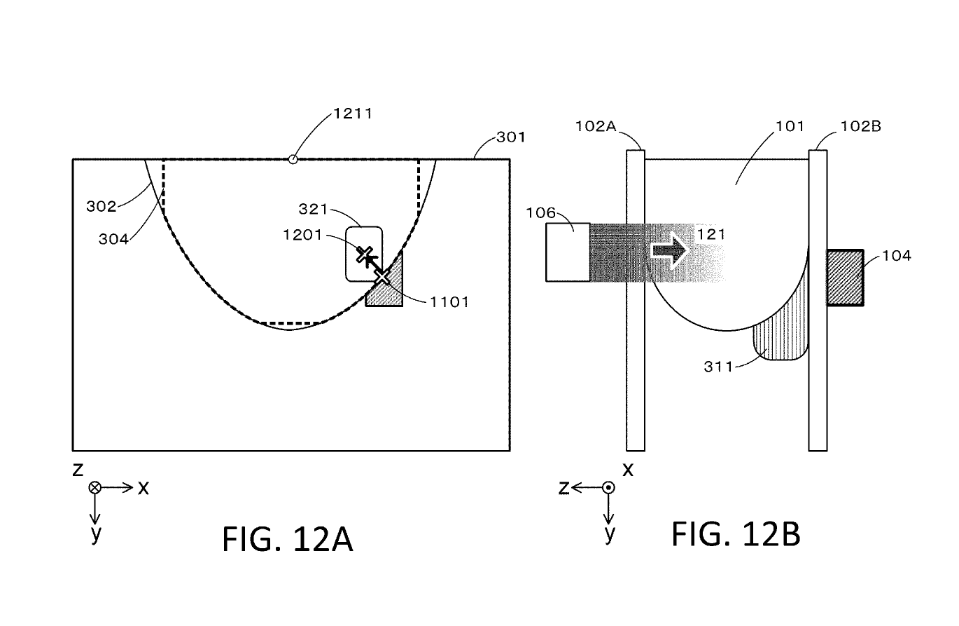

Subsequently, by using FIGS. 11 and 12, a description will be given of control performed in the case where the acquisition position of the object information does not fall within the light irradiation area in the determination in Step S903. Herein, the acquisition of the object information corresponding to the object shape is performed.

Similarly to FIG. 10, each of FIGS. 11A and 12A is a front view of the held object 101 as viewed from the side of the holding plate 102A. In addition, each of FIGS. 11B and 12B is a side view thereof.

In FIG. 11, an acquisition position 1101 of the object information specified by the user is positioned in the vicinity of the peripheral edge part of the object 101. Accordingly, the shape 321 of the pulsed light 121 does not fall within the light irradiation area 304. When the basic scanning control is performed in this state, as shown in FIGS. 11A and 11B, a part of the pulsed light 121 reaches the surface of the probe 104 while maintaining high energy. As a result, the noise resulting from the strong photoacoustic wave generated by the surface of the probe 104 appears in the reconstructed image.

In the case where the acquisition position of the object information does not fall within the light irradiation area 304, as shown in FIG. 12A, the control processor 111 corrects the irradiation position of the pulsed light 121 from the position 1101 specified by the user to a position 1201. Subsequently, the control processor 111 generates the position control information required for the movement to the position 1201.

At the corrected position 1201, the light shape 321 falls within the light irradiation area 304. Under this premise, an area of overlap between the light shape 321 and the probe 104 is maximized. Note that the position of the probe 104 denotes the area of the probe 104 projected on a two-dimensional plane formed at the boundary between the holding plate 102A and the object 101 to be precise.

By setting the corrected position at the position where the area of overlap is maximized, it is possible to receive the photoacoustic wave from the object area positioned at the front of the probe 104 while maintaining high energy efficiency.

Note that, at the corrected position 1201, as shown in FIGS. 12A and 12B, a part of the light 121 does not reach the probe 104 directly (i.e., while maintaining high energy without going through the object). Accordingly, by reconstructing the object information using the photoacoustic wave signal, it is possible to acquire the image in which the noise is reduced.

In FIG. 12, the position at which the area of overlap between the light shape 321 and the probe 104 is maximized is specified as the corrected position. However, by using a line segment joining the position 1101 specified by the user and any position 1211 specifying a correction direction newly specified on the object as the correction direction, the corrected position may be set on the line segment.

In Step S906, the position control section 112 acquires the photoacoustic wave from the object area corresponding to an acoustic element arrangement area of the probe 104 according to the position control information generated in Step S904 or Step S905. Since the probe 104 does not scan in the present embodiment, the acoustic element arrangement area can be regarded as a reception aperture area.

Processing in subsequent Steps S907 to S908 is the same as that in Steps S207 to S208 in FIG. 2. With this, the reconstructed object information is generated as image data and displayed.

With the object information acquiring method described above, it is possible to correct the irradiation position of the pulsed light 121 correspondingly to the shape of the object 101 relative to the specified acquisition position of the object information and acquire the photoacoustic wave. With this, it is possible to suppress the strong photoacoustic wave generated by the surface of the probe 104 that becomes manifest as the noise when the object information is visualized.

### Third Embodiment

A third embodiment that uses the object information acquiring method of the present invention will be described according to the drawings.

In the first and second embodiments, the scanning control of the irradiation position of the pulsed light and the scanning control of the probe reception position are individually performed, and the selective control is performed such that the irradiation position of the pulsed light and its scanning to the object 101 fall within the object shape. With this, the pulsed light is prevented from directly reaching the probe.

In the present embodiment, the acquisition of the object information is performed such that the pulsed light does not directly reach the probe only by controlling the irradiation position of the pulsed light and the reception position of the probe irrespective of the object shape. With this, even in the case where the object shape is small or in the case where it is relatively difficult to use the object shape at a position such as the peripheral edge part of the object having a curved shape, similar effects are obtained.

Hereinbelow, characteristic parts of the present embodiment will be mainly described.

### (Component and Function)

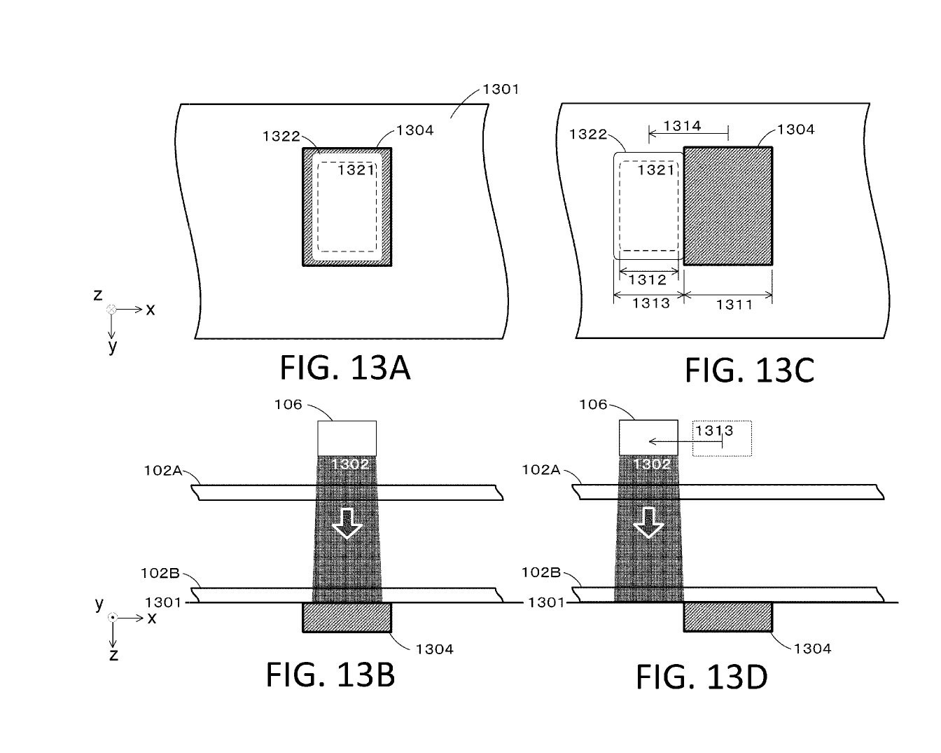

FIG. 13 is a conceptual view for explaining the object information acquiring method in the third embodiment.

Each of FIGS. 13A to 13D shows the positional relationship between the irradiation position of the pulsed light 121 and the reception position of a probe 1304 controlled to be positioned at any positions of the holding plate 102 as the movement surface of the position control (or the scanning surface of the two-dimensional scanning). FIGS. 13A and 13B show the conventional method, while FIGS. 13C and 13D show the object information acquiring method in the present embodiment. Note that an xy plane shown in FIGS. 13A and 13C shows a cross section in a movement surface 1301 of the probe 1304, and the size of a light shape 1322 or the probe 1304 is indicated by a projected image on the cross section 1301.

The reference numeral 1321 indicates the light shape of a pulsed light 1302 on the xy plane at the emission end of the irradiation optical system 106. In FIG. 13, the pulsed light 1302 has a constant enlargement tendency in its travelling direction, i.e., in a z-axis direction. As a result, a light shape 1322 on the surface 1301 is larger.

In FIGS. 13A and 13B, position control is performed such that the light shape 1321 (or 1322) and the probe 1304 maintain the opposing relationship and the center positions thereof match each other. In such a positional relationship, in the case where the object is not present between the holding plates 102, the pulsed light 1302 reaches the surface of the probe 1304 while maintaining high energy without being decayed. As a result, the surface of the probe generates the strong photoacoustic wave.

In contrast to this, as shown in FIGS. 13C and 13D, by moving the irradiation position of the pulsed light 1302 in an x-axis direction by the reference numeral 1314, it is possible to prevent the pulsed light 1302 from directly reaching the probe 1304. The position control amount 1314 may be calculated by adding a width 1311 of the probe 1304 and a width 1313 of a projected light shape 1322 in the x-axis direction together and dividing the value obtained by the addition by 2.

In the case where the light 1302 is ideal parallel light, a position control amount 1313 may be calculated by adding the width 1311 and a width 1312 together and dividing the value obtained by the addition by 2. In addition, in the case where the light 1302 has a reduction tendency as well, the position control amount 1313 can be calculated by the similar calculation.

Although the position control in the x-axis direction is performed on the irradiation position of the light 121 in FIG. 13, the position control in the y-axis direction may also be performed and, even when the position control is performed on the probe 1304, similar effects can be obtained.

In addition, although the movement surface of the probe 1304 is selected as the projected cross section for the comparison between the light irradiation position and the probe reception position in FIG. 13, the cross section orthogonal to the travelling direction of the light 121 or the direction of the normal to the surface of the probe may be appropriately set arbitrarily.

The embodiment in the case where the holding plates 102 are configured by parallel planes and the pulsed light 1302 and the probe 1304 maintain the opposing relationship is described in FIG. 13. However, even in the case where the direction of emission of the pulsed light 1302 is angled, the position control can be similarly performed by projecting the light irradiation shape and the reception area (reception surface shape) of the probe on any cross section and comparing their positions with each other. Further, the same applies to the case where the surface of the probe is set so as to be inclined relative to the movement surface.

In the case where at least one of the holding plates 102 has a shape having a curvature and at least one of the movement surface (i.e., the scanning surface) of the light irradiation position along the holding plate and the movement surface of the probe has a curvature, the position control can be performed by comparing the positions using the projected images on any cross section.

### (Time-Series Operation of Selective Scanning Control)

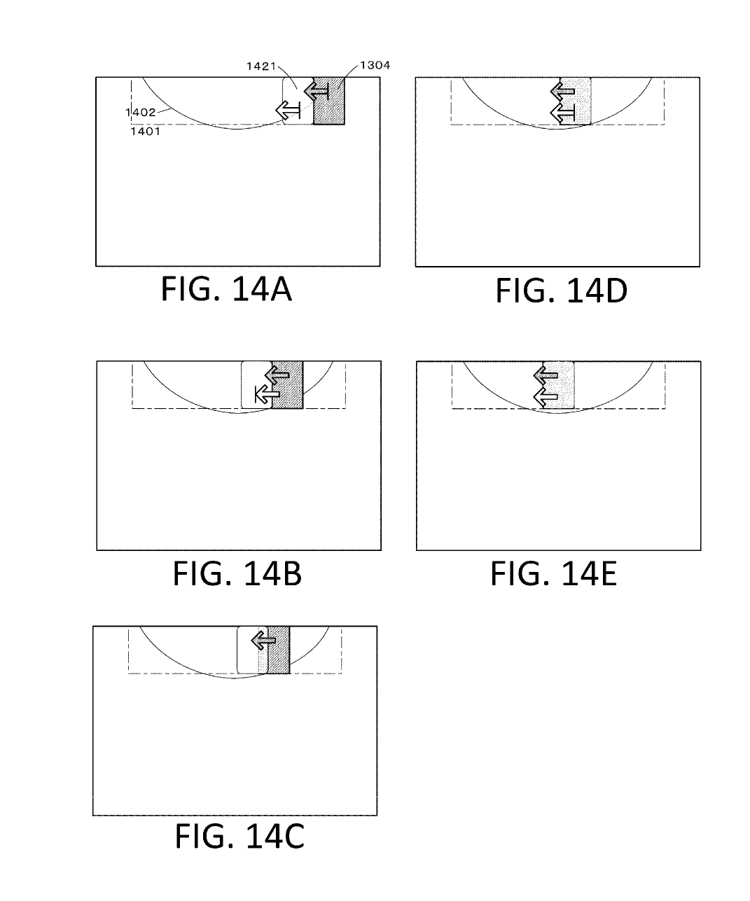

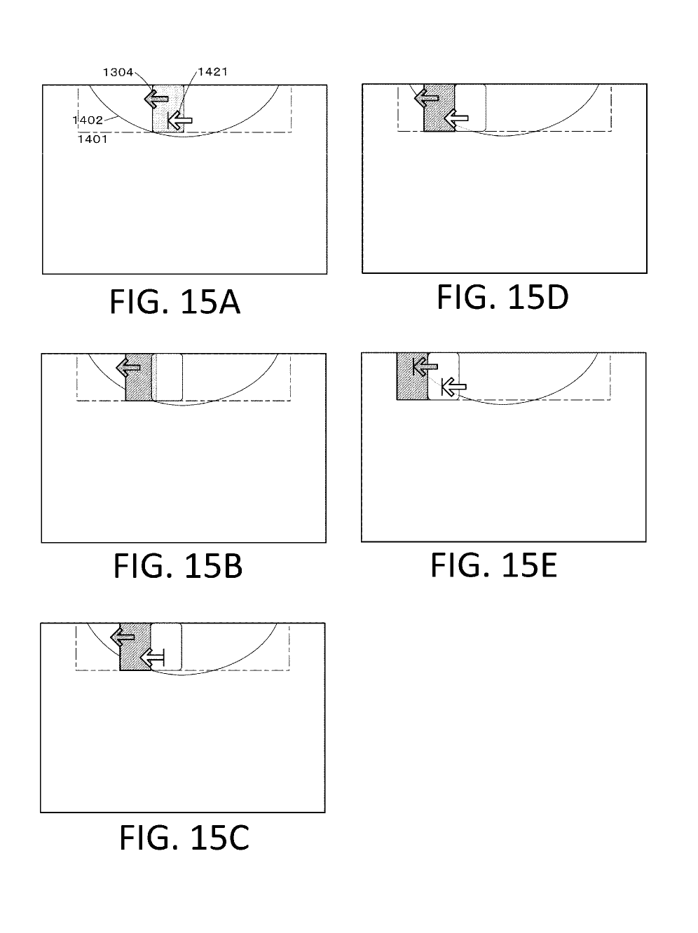

Subsequently, by using FIGS. 14 and 15, a description will be given of the time-series operation of the scanning control during the acquisition of the object information in the third embodiment.

FIGS. 14A to 14E and FIGS. 15A to 15E show the time-series operations of the two-dimensional scanning using the irradiation position of the pulsed light 1421 and the probe 1304 to an acquisition region 1401 of the object information specified by the user. The reference numeral 1402 indicates the outermost outline of the object. Pulsed light 1421 in FIGS. 14 and 15 is assumed to have the light shape similar to that of the probe 1304 and is assumed to be ideal parallel light for the sake of the description.

FIG. 14A shows a state in which the pulsed light 1421 and the probe 1304 have moved to the respective scanning start positions in synchronization with the acquisition start of the object information. As described in connection with FIG. 13, according to the third embodiment, also by disposing the irradiation position of the pulsed light at a position close to the probe 1304, it is possible to prevent the light from directly reaching the probe 1304.

The position control section 112 moves the position of the pulsed light 1421 and the probe 1304 simultaneously from the positions in FIG. 14A to thereby start the main scanning.

Thereafter, when the positions in FIG. 14B are reached, the light irradiation position control section 112A stops the movement of the pulsed light 1421. At this position, the pulsed light 1421 completely falls within the object shape 1402, and hence all of its energy is inputted into the object. A part or all of the pulsed light 1421 is prevented from reaching the probe 1304 while maintaining high energy, and it is possible to acquire the photoacoustic wave from the peripheral edge part of the object 1402.

In FIG. 14C, the probe position control section 112B continues the main scanning of the probe 1304. During this operation, the light irradiation position control section 112A repeats the irradiation of the pulsed light 1421 to the object 1402 while keeping the position of the pulsed light 1421 at the same position. The pulsed light 1421 waits until the probe 1304 reaches this position.

Next, as shown in FIG. 14D, the light irradiation position control section 112A starts the drive of the movement mechanism to resume the main scanning in synchronization with the arrival of the probe 1304 at the wait position of the pulsed light 1421. After the pulsed light and the probe overlap one another, as shown in FIG. 14E, the main scanning is performed while the opposing relationship between the pulsed light 1421 and the probe 1304 is maintained.

When the positions of FIG. 15A are reached as the result of the continuation of the main scanning, the light irradiation position control section 112A stops the main scanning of the pulsed light 1421. This is because a part of the pulsed light 1421 reaches the probe 1304 directly when the pulsed light 1421 moves past this position while the opposing relationship is maintained. The probe position control section 112B continuously performs the main scanning of the probe 1304.

In FIG. 15B, the probe position control section 112B continues the main scanning of the probe 1304. During this operation, similarly to the case in FIG. 14C, the light irradiation position control section 112A repeats the irradiation of the pulsed light 1421 to the object 1402 while keeping the position of the pulsed light 1421 at this position. The pulsed light 1421 waits until the probe 1304 reaches this position.

When the probe 1304 reaches the position of FIG. 15C, the light irradiation position control section 112A resumes the main scanning of the pulsed light 1421. In the positional relationship between the pulsed light 1421 and the probe 1304 shown in FIG. 15C, a part or all of the pulsed light 1421 does not reach the probe 1304 directly.

Thereafter, the position control section 112 continues the main scanning of each of the pulsed light 1421 and the probe 1304 while maintaining the positional relationship shown in FIG. 15D and, when the positions of FIG. 15E are reached, the acquisition of the object information is completed.

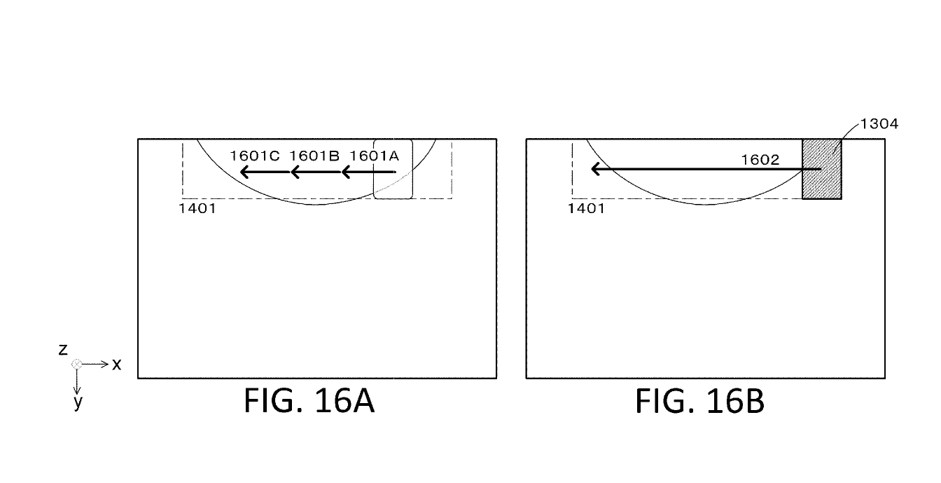

FIG. 16 is a conceptual view for explaining the scanning control in the third embodiment, and shows the scanning track of the time-series operations of FIGS. 14 and 15. FIG. 16A shows the scanning track of the pulsed light 1421 to the acquisition region 1401 of the object information.

As shown in FIG. 16B, with regard to the probe 1304, one main scanning 1602 is successively performed. At this point, by scanning control in which three main scannings represented by three scanning lines 1601A to 1601C and stop periods between them are combined, the pulsed light 1421 is selectively emitted to the object 1402. With this, it is possible to prevent the pulsed light 1421 from directly reaching the probe 1304.

With the above-described method, in the measurement of the photoacoustic wave in the case where the object shape is small or in the peripheral edge part of the object shape, the irradiation position of the pulsed light and the probe reception position are not spaced apart farther than necessary, and it is possible to acquire the object information while maintaining high use efficiency of light energy.

In the first embodiment, since only the selective scanning control is performed such that the irradiation position of the pulsed light and its scanning fall within the object shape, the peripheral edge part of the object shown in FIG. 7C is created. However, even in such a case, according to the present embodiment, it is possible to keep the irradiation position of the pulsed light and the reception position of the probe close to each other, and acquire the object information with high efficiency.

### Fourth Embodiment

In addition, the object of the present invention is also achieved by the following configuration. That is, a storage medium (or a recording medium) that stores the program code of software for implementing the functions of the above-described embodiments is supplied to a system or an apparatus. Subsequently, a computer (or a CPU or an MPU) of the system or the apparatus reads and executes the program code stored in the storage medium.

In this case, the program code read from the storage medium implements the functions of the above-described embodiments, and the storage medium storing the program code constitutes the present invention.

In addition, by executing the program code read by the computer, an operating system (OS) operating on the computer performs a part or all of actual processing based on the instruction of the program code. It goes without saying that the case where the functions of the above-described embodiments are implemented by the processing is included in the scope of the present invention.

Further, it is assumed that the program code read from the storage medium is written in a memory provided in a function extension card inserted into the computer or a function extension unit connected to the computer. It goes without saying that the case where the CPU provided in the function extension card or the function extension unit performs a part or all of the actual processing based on the instruction of the program code and the functions of the above-described embodiments are implemented by the processing is included in the scope of the present invention.

In the case where the present invention is applied to the above storage medium, the program code corresponding to the above-described flowchart is stored in the storage medium.

### Other Embodiments

A person skilled in the art can easily conceive of configuring a new system by appropriately combining various technologies in the above-described embodiments. Consequently, the system configured by various combinations also belongs to the scope of the present invention.

In addition, in each of the embodiments described above, the configuration is adopted in which the irradiation position or the irradiation area of the pulsed light is selectively controlled. However, also in the configuration in which the reception position or the reception aperture area of the probe is selectively controlled, similar effects can be obtained. That is, by moving the probe to a position into which light does not enter as well, the object of the present invention can be achieved.

In this case, the control processor 111 generates the control information of the reception position or the reception scanning area of the probe 104 correspondingly to the acquired object shape. Alternatively, in the case where the acquisition of the object information is performed by using the probe larger than the object shape, selective reception control may be appropriately performed on each of the plurality of the acoustic elements constituting the probe. It is also preferable to perform the selective reception control to thereby control the reception aperture area. With the configuration described above, the control processor 111 can selectively control the reception position or the reception aperture area of the probe.

In this case, the photoacoustic wave is received at a position or an area different from the acquisition position of the object information specified by the user. However, when the target object information is visualized, imaging may be appropriately performed with the object area desired by the user selected as the target.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-188242, filed on Sep. 11, 2013, which is hereby incorporated by reference herein in its entirety.

What is claimed is:

1. An object information acquiring apparatus comprising:
an irradiating unit configured to irradiate an object with light;
an irradiation position controlling unit configured to control an irradiation position for irradiating the object with the light;
a probe configured to receive an acoustic wave generated when the object is irradiated with the light from the irradiating unit, at a position substantially opposing the irradiating unit across the object, and output an acoustic wave signal;
a probe controlling unit configured to control reception of the probe;
a control processor configured to control at least one of the irradiation position controlling unit and the probe controlling unit such that the light does not enter the probe directly without going through the object; and
a constructing unit configured to construct characteristic information on an inside of the object from the acoustic wave signal.

2. The object information acquiring apparatus according to claim 1, wherein
the control processor controls at least one of the irradiation position controlling unit and the probe controlling unit in a case where projected images of a distribution shape of the light and a reception area of the probe on any cross section overlap one another.

3. The object information acquiring apparatus according to claim 1, wherein
the control processor controls position of the irradiating unit such that the light selectively scans the object.

4. The object information acquiring apparatus according to claim 3, wherein
the control processor controls position of the irradiating unit such that a projection image of the light on the object scans an area inside an outermost outline of the object according to a irradiating direction and a distribution shape of the light at the point of irradiation.

5. The object information acquiring apparatus according to claim 1, wherein
the probe includes a plurality of acoustic elements arranged along at least a first direction, and
the probe controlling unit is configured to control a reception opening by selectively performing reception control on the plurality of acoustic elements constituting the probe, and
the control processor controls a reception aperture area of the probe.

6. The object information acquiring apparatus according to claim 1, wherein
the probe controlling unit is configured to control a receiving position of the probe, and
the control processor controls a position of the probe during scanning of the probe.

7. The object information acquiring apparatus according to claim 6, wherein
the control processor controls the irradiation position controlling unit and the probe controlling unit such that the light and the probe scan in synchronization with each other.

8. The object information acquiring apparatus according to claim 7, wherein
the control processor causes the probe to scan while keeping the irradiation position on the object in a case where a positional relationship allowing the light to enter the probe directly without going through the object is established as a result of the synchronized scanning of the light and the probe, such that a shape of the light projected on surface of the object falls within a shape of the object.

9. The object information acquiring apparatus according to claim 1, further comprising
an operating unit that receives an operation for specifying an area of the object constituting the characteristic information, wherein
the control processor calculates an irradiation area irradiated with the light based on the specified area, generates irradiation position control information, and, outputs the irradiation position control information to the irradiation position controlling unit.

10. The object information acquiring apparatus according to claim 9, further comprising
a shape acquiring unit configured to acquire shape information on the object, wherein
the control processor generates the irradiation position control information based on the shape information and the irradiation area and outputs the irradiation position control information to the irradiation position controlling unit.

11. The object information acquiring apparatus according to claim 10, wherein
the probe has a function of transmitting an ultrasonic wave to the object and receiving an ultrasonic echo reflected in the object, and
the shape acquiring unit acquires the shape information by using the ultrasonic echo.

12. The object information acquiring apparatus according to claim 10, wherein
the shape acquiring unit acquires the shape information by using an image obtained by imaging the object.

13. The object information acquiring apparatus according to claim 2, wherein
the control processor controls the irradiation position controlling unit and the probe controlling unit such that an area of overlap between the projected images of the distribution shape of the light and the reception area of the probe is maximized.

14. The object information acquiring apparatus according to claim 1, further comprising
two holding plates configured to hold the object, wherein
the irradiating unit and the probe are disposed on the different holding plates, respectively.

15. The object information acquiring apparatus according to claim 1, further comprising
a displaying unit configured to display the characteristic information.

16. A control method of an object information acquiring apparatus having an irradiating unit, an irradiation position controlling unit, a probe, a probe controlling unit, a control processor, and a constructing unit, the control method comprising:
an irradiating step in which the irradiating unit irradiates an object with light;
an irradiation position controlling step in which the irradiation position controlling unit controls an irradiation position for irradiating the object with the light;
a receiving step in which the probe receives an acoustic wave generated when the object is irradiated with the light from the irradiating unit, at a position opposing the irradiating unit across the object, and outputs an acoustic wave signal;
a probe controlling step in which the probe controlling unit controls the probe when the probe receives the acoustic wave;
a controlling step in which the control processor controls at least one of the irradiation position controlling unit and the probe controlling unit such that the light does not enter the probe directly without going through the object; and
a constructing step in which the constructing unit constructs characteristic information on an inside of the object from the acoustic wave signal.
